(19) United States
(12) Patent Application Publication
(10) Pub. No.: US 2017/0196816 A1
(43) Pub. Date: Jul. 13, 2017

(54) LASER-PRINTABLE TABLET, AND METHOD FOR MANUFACTURING THE SAME

(71) Applicant: NIPRO CORPORATION, Osaka-shi, Osaka (JP)

(72) Inventors: Yohei HOASHI, Osaka-shi, Osaka (JP); Shotaro IKUTA, Osaka-shi, Osaka (JP)

(73) Assignee: NIPRO CORPORATION, Osaka-shi, Osaki (JP)

(21) Appl. No.: 15/315,449

(22) PCT Filed: Jun. 2, 2015
(86) PCT No.: PCT/JP2015/065870

§ 371 Date: Dec. 1, 2016

(30) Foreign Application Priority Data
Jun. 2, 2014 (JP) 2014-113781

(51) Int. Cl. A61K 9/20; A61K 9/28; A61K 31/47; A61J 3/00

(52) U.S. Cl. CPC A61K 9/2072; A61J 3/005; A61K 9/2893; A61K 9/2886; A61K 9/2813; A61K 9/2866; A61K 31/47

(57) ABSTRACT

A laser-printable tablet has: a tablet containing an optically unstable drug, a first cover layer that covers the tablet and contains a first color change-inducing oxide, and a second cover layer that covers the first cover layer and contains a second color change-inducing oxide, the concentration in which the second color change-inducing oxide is contained in the second cover layer being different from the concentration in which the first color change-inducing oxide is contained in the first cover layer.

### TECHNICAL FIELD

The present invention relates to a laser-printable tablet and to a method for its manufacture. More precisely, it relates to a tablet that contains a drug that is optically unstable but can be laser printed, and to a method for manufacturing this tablet.

### BACKGROUND ART

In order to reduce the risk of treatment error with tablets that are handled in a medical setting, before the tablets are provided to patients, the writing on the tablet surface is read and checked against a code table to identify the product name and so forth, confirming that the proper tablets are being given to the patients.

Gravure offset printing and inkjet printing are methods for printing letters and numbers on the surface of a tablet, but problems were encountered with these printing methods. Specifically, since ink was used, there was sometimes poor printing contrast, ink that had yet to dry could rub off on other tablets, the writing could become smudged, and so forth, and in addition to problems such as these, there were also problems related to the environment since ink and organic solvents were used. WO 2006/126561, for example, discloses a UV laser printing technique as a printing method that avoids such problems.

UV laser printing is a technique in which a coating film containing a particular color change-inducing oxide (titanium oxide, yellow iron sesquioxide, and iron sesquioxide) is formed on the surface of the tablet, and the tablet is irradiated with a particular laser beam to print distinct identification writing. Patent Literature 1 discloses that it is preferable for 100 weight parts of coating film to contain 0.01 to 20 weight parts color change-inducing oxide.

An attempt to improve the clarity of writing produced by UV laser printing has been disclosed in Japanese Laid-Open Patent Application 2013-155148, for example, as another prior art UV laser printing technique. Also, Japanese Laid-Open Patent Application 2014-47161 discloses a technique for making the coating film into a thin film.

### SUMMARY OF INVENTION

### Technical Problem

However, in the case that a drug that is optically unstable is contained as a component of a tablet, when the tablet is irradiated with laser light, and when the tablet is irradiated with natural light such as sunlight or fluorescent light, due to the decomposition of the drug in the tablet, the effective ingredient of the drug can lose its function. To protect the drug against such decomposition, it is possible to increase the amount in which the color change-inducing oxide is contained in the cover layer, but if the proportion of the color change-inducing oxide in the cover layer is too high, the tensile strength of the coating film will decrease, which is a problem in that the coating film may crack or chip in the course of tablet production, packaging, shipping, dividing, etc.

The present invention was conceived in light of the above problems, and it is an object thereof to provide a laser-printable tablet with which writing that is easy to identify can be printed on the surface of the tablet, and a drug that is optically unstable can be protected against exposure to laser light.

### Solution to Problem

The inventors conducted painstaking study into the composition and configuration of a coating film that affords easy printing on the surface of a tablet and that avoids the decomposition of the drug due to irradiation with natural light or a laser beam, which led to the finding that if a tablet surface is given a coating of two layers with different compositions and/or amounts of a color change-inducing oxide, it will be easy to print on the tablet surface and at the same time the drug can be protected. They then conducted painstaking study into the optimal amount in which the color change-inducing oxide is contained in each layer on the basis of this finding, and this led to the perfection of the present invention as set forth below.

Specifically, (1) the laser-printable tablet of the present invention has a tablet containing an optically unstable drug, a first cover layer that covers the tablet and contains a first color change-inducing oxide, and a second cover layer that covers the first cover layer and contains a second color change-inducing oxide, wherein the concentration in which the second color change-inducing oxide is contained in the second cover layer is different from the concentration in which the first color change-inducing oxide is contained in the first cover layer.

(2) It is preferable if the first cover layer contains the first color change-inducing oxide in an amount of at least 10 wt % and no more than 50 wt %, and the concentration in which the second color change-inducing oxide is contained in the second cover layer is lower than the concentration in which the first color change-inducing oxide is contained in the first cover layer.

(3) It is preferable if the second cover layer contains the second color change-inducing oxide in an amount of no more than 15 wt %.

(4) It is preferable if the first cover layer contains the first color change-inducing oxide in an amount of at least 15 wt % and no more than 30 wt %.

(5) It is preferable if the optically unstable drug is one or more types selected from the group including of olanzapine, montelukast sodium and rosuvastatin.

(6) The method for manufacturing a laser-printable tablet of the present invention has a step of covering the surface of a tablet containing an optically unstable drug with a first coating agent containing a first color change-inducing oxide, a step of covering the surface of the first coating agent with a second coating agent containing a second color change-inducing oxide whose concentration is different from that of the first color change-inducing oxide, and a step of drying the first coating agent and the second coating agent, thereby forming a first cover layer and a second cover layer in that order on the surface of the tablet.

(7) It is preferable if the first coating agent contains a color change-inducing oxide in an amount of at least 10 wt % and no more than 50 wt % with respect to the total solids content, and the concentration of the second color change-inducing oxide contained in the second coating agent is lower than the concentration of the first color change-inducing oxide contained in the first coating agent.

### Advantageous Effects of Invention

The laser-printable tablet of the present invention has an outstanding effect in that it allows writing to be easily printed on the surface of a tablet, and can protect an optically unstable drug in the tablet against exposure to laser light.

### BRIEF DESCRIPTION OF DRAWINGS

FIG. 1 is a simplified cross section of the laser-printable tablet of the present invention;

FIG. 2a is a detail photograph of the writing after the laser-printable tablet in Example 1 has been UV printed, and FIG. 2b is a detail photograph of the writing after the laser-printable tablet in Comparative Example 1 has been UV printed; and

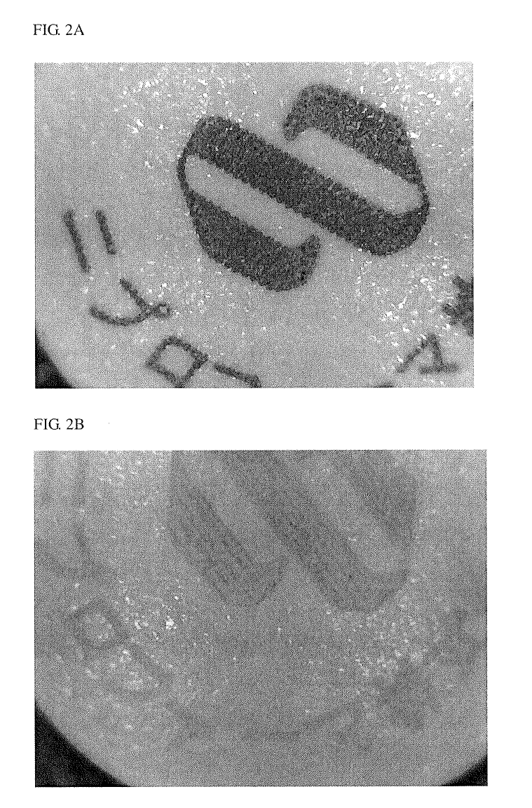

FIG. 3a is a detail photograph of a place where a small crack or chip has formed in the surface of a tablet (light damage), and FIG. 3b is a detail photograph of a place where a more pronounced crack or a chip has formed in the surface of the tablet (heavy damage).

### DESCRIPTION OF EMBODIMENTS

### Laser-Printable Tablet

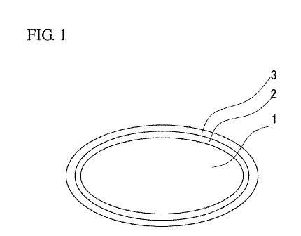

FIG. 1 is a simplified cross section of the laser-printable tablet of the present invention. As shown in FIG. 1, the laser-printable tablet of the present invention has a tablet 1 containing an optically unstable drug, a first cover layer 2 containing a first color change-inducing oxide, and a second cover layer 3 containing a second color change-inducing oxide. The characteristic feature is that the concentration in which the second color change-inducing oxide is contained in the second cover layer 3 is different from the concentration in which the first color change-inducing oxide is contained in the first cover layer.

By using two types of cover layer with different concentrations of the color change-inducing oxide as discussed above, the cover layer with the higher concentration will ensure the protection of the drug when it is irradiated with laser light, while the cover layer with the lower concentration will improve the durability of the tablet and printing by laser beam. Consequently, the printability of the laser-printable tablet, the drug protection function, and durability can all be improved. In FIG. 1, a two-layer structure consisting of the first cover layer 2 and the second cover layer 3 is shown, but as long as a first cover layer and a second cover layer are included, the laminate structure may have three or more layers, and there are no particular restrictions on the maximum number of cover layers. The color change-inducing oxide may or may not be contained in a cover layer other than the first cover layer and second cover layer. The concentration of the color change-inducing oxide if it is contained is preferably lower than the concentrations of the color change-inducing oxides contained in the first cover layer and the second cover layer. The compositions of the color change-inducing oxides may be the same or different. The term “composition” as used herein means the type and/or proportion (concentration) of the substance.

It is preferable if the first cover layer 2 contains the first color change-inducing oxide in an amount of at least 10 wt % and no more than 30 wt %, and the concentration in which the second color change-inducing oxide is contained in the second cover layer is lower than the concentration in which the first color change-inducing oxide is contained in the first cover layer. Since the second cover layer 3 makes up the surface of the tablet, this is because it is preferable to lower the concentration of the color change-inducing oxide and improve durability. Aside from having different concentrations, the first color change-inducing oxide and second color change-inducing oxide here preferably are the same compound, but may instead be different types of compound. If a plurality of compounds are contained, they may have the same or different compositions. The concentration of each compound may be such that the total amount of the first color change-inducing oxide and the total amount of the second color change-inducing oxide have the above-mentioned relation.

The various components of the laser-printable tablet of the present invention will be described below.

Tablet

In the present invention, the tablet 1 contains an optically unstable drug and additives. There are no particular restrictions on the shape of the tablet 1, which can be in the form of a disk, a donut, a polygonal plate, a sphere, an ellipse, a caplet, or the like, but a disk shape like that of an ordinary tablet is preferable. There are no particular restrictions on the size of the tablet 1, but preferably the diameter is 3 to 30 mm and the thickness is 1 to 10 mm, for example.

The “optically unstable drug” in the tablet here means that when the drug is exposed to light, using outdoor daylight as set forth in ISO10977 (1993) with a total luminance of at least 1,200,000 lux·hr or light with a total near ultraviolet irradiation energy of at least 200 W·h/m² (preferably, light with a total luminance of at least 1,200,000 lux·hr and a total near ultraviolet irradiation energy of at least 200 W·h/m²), the drug decomposes enough to require confirmation of toxicity and/or identification of the structural formula of impurities (decomposition products) produced by the decomposition of the drug. Whether or not it is necessary to confirm toxicity or the structural formula is difficult to establish unconditionally since it will vary with the properties and/or toxicity of the decomposition products, but in general, the toxicity and/or the structural formula of the decomposition products will need to be confirmed when the amount of impurities (decomposition products) produced exceeds 0.1 wt % with respect to the drug. Also, even if the amount in which the impurities (decomposition products) are produced is within the above range, a drug that changes color overall enough to be visible to visual when exposed to the above-mentioned daylight will also fall under the heading of “optically unstable drug.”

There are no particular restrictions on this optically unstable drug, but examples include one or more types of olanzapine, montelukast sodium, amlodipine, donepezil, ebastine, selegiline, famotidine, irsogladine, brotizolam, olanzapine, lansoprazole, bepotastine, ramosetron, tamsulosin, naftopidil, porapurezinc, voglibose, rizatriptan, midodrine, risperidone, ondansetron, loratadine, montelukast, azulenesulfonate acid, etizolam, enalapril, captopril, glibenclamide, chlormadinone acetate, doxazosin, triazolam, domperidone, ketotifen, bromperidol, pravastatin, simvastatin, pravastatin, rosuvastatin, atorvastatin, loperamide, lisinopril, rilmazafone, mecobalamin, alfacalcidol, bromocriptine and pramipexole as well as pharmaceutically acceptable salts and solvates thereof.

There are no particular restrictions on the additives in the tablet, and any of those normally used in the medical field may be used, examples of which include excipients, disintegrating agents, lubricants, binders, dissolution auxiliaries, plasticizers, sweeteners, flavorings, foaming agents, surfactants, preservatives, and the like, which may each be used alone, or two or more types may be used together.

First Cover Layer

The first cover layer 2 covers the surface of the tablet, and usually has a thickness of at least 1 μm and no more than 100 μm, although there are no particular restrictions on the thickness. The concentration of the first color change-inducing oxide contained in the first cover layer 2 is different from the concentration of the second color change-inducing oxide contained in the second cover layer 3. The first cover layer 2 preferably contains at least 50 wt % and no more than 90 wt % film component and at least 10 wt % and no more than 50 wt % first color change-inducing oxide. Having the color change-inducing oxide be contained in this proportion allows the first cover layer 2 to block natural light and laser light and thereby protect the drug from decomposition. In the case that too much color change-inducing oxide is used, the surface of the tablet will be prone to cracking and chipping, but too little is used, it will not be able to block natural light or laser light, and the drug will decompose. The color change-inducing oxide contained in the first cover layer 2 is preferably contained in an amount of at least 12 wt % and no more than 40 wt %, more preferably at least 15 wt % and no more than 30 wt %, and particularly preferably at least 18 wt % and no more than 28 wt %.

The term “color change-inducing oxide” here means an oxide that induces a color change upon irradiation with laser light. The color change-inducing oxide is one or more types of compound selected from the group including titanium oxide, yellow iron sesquioxide, and iron sesquioxide. The type, wavelength, output, and so forth of the laser light here can be suitably adjusted in order to induce the intended color change.

Examples of the film component in the first cover layer 2 include methyl cellulose, hydroxypropyl cellulose, low-substituted hydroxypropyl cellulose, hypromellose, croscarmellose sodium, dextrin, pullulan, gum arabic, agar, gelatin, tragacanth, sodium alginate, povidone, polyvinyl alcohol, paraffin, microcrystalline wax, cetyl alcohol, stearyl alcohol, stearic acid, sorbitan fatty acid ester, glyceryl monostearate, macrogol 400, macrogol 600, macrogol 4000, macrogol 6000, macrogol 20000, and the like.

The first cover layer 2 may contain any additive used in the medical field, as a ingredient other than the film component and the first color change-inducing oxide. Examples of such additives include excipients, disintegrating agents, lubricants, binders, dissolution auxiliaries, plasticizers, sweeteners, flavorings, foaming agents, surfactants, and preservatives.

Second Cover Layer

The second cover layer 3 covers the surface of the first cover layer 2, and usually has a thickness of at least 1 μm and no more than 100 μm, although there are no particular restrictions on the thickness. The concentration of the second color change-inducing oxide contained in the second cover layer 3 is preferably lower than the concentration of the first color change-inducing oxide contained in the first cover layer 2. Reducing the concentration of the color change-inducing oxide improves the durability of the second cover layer 3, and therefore the durability of the tablet itself can be improved. The second cover layer 3 preferably contains the second color change-inducing oxide in an amount of no more than 15 wt %, and more preferably contains the second color change-inducing oxide in an amount of no more than 10 wt %, and even more preferably contains the second color change-inducing oxide in an amount of no more than 8 wt %. From the standpoint of improving printing performance under irradiation with laser light, the color change-inducing oxide is preferably contained in the second cover layer in an amount of at least 0.5 wt % and no more than 10 wt %, and more preferably at least 1 wt % and no more than 5 wt %.

The second cover layer is similar to the first cover layer in that it may contain a film component and other additives. Examples of the film component and additives are the same as those listed for the first cover layer.

Method for Manufacturing Laser-Printable Tablet

The method of the present invention for manufacturing a laser-printable tablet includes a step of covering the surface of a tablet containing an optically unstable drug with a first coating agent, a step of covering the surface of the first coating agent with a second coating agent, and a step of drying the first coating agent and the second coating agent, thereby forming a first cover layer and a second cover layer in that order on the surface of the tablet. It is a characteristic feature that the concentration in which the color change-inducing oxide is contained in the first coating agent is different from the concentration (wt %) of the color change-inducing oxide contained in the second coating agent. With the laser-printable tablet produced in this way, it is easy to print writing on the surface of the tablet, and an optically unstable drug in the tablet can be protected against exposure to laser light.

It is preferable that the first coating agent contains a first color change-inducing oxide in an amount of at least 10 wt % and no more than 50 wt % with respect to the total solids content, and the concentration of the second color change-inducing oxide contained in the second coating agent is lower than the concentration of the first color change-inducing oxide contained in the first coating agent. Using coating agents such as these allow the formation of a first cover layer and second cover layer containing specific concentrations of color change-inducing oxide.

Step of Covering with First Coating Agent

Firstly, the surface of the tablet is covered with the first coating agent. The first coating agent preferably contains an adjusting solvent, the purpose of which is to adjust to a viscosity that is suited to spraying. Examples of the adjusting solvent include water, ethanol, and methanol. There are no particular restrictions on how the surface of the tablet is covered with the first coating agent, so long as it can be covered, but covering by spraying is preferable. The solids of the first coating agent have the same constitution as the component forming the first cover layer. Therefore, the first coating agent preferably contains the first color change-inducing oxide in an amount of at least 10 wt % and no more than 50 wt % with respect to the total solids content.

Step of Covering with Second Coating Agent

Next, the surface of the tablet covered by the first coating agent is covered with the second coating agent. The concentration in which the second color change-inducing oxide is contained in the second coating agent is preferably lower than the concentration in which the first color change-inducing oxide is contained in the first coating agent. This allows the formation of a second cover layer with a lower concentration of color change-inducing oxide than the first cover layer. The solids of the second coating agent have the same constitution as the component forming the second cover layer. The second coating agent preferably contains less than 10 wt % color change-inducing oxide with respect to the total solids content. The adjusting solvent contained in the second coating agent may be different from the one contained in the first coating agent, but it is preferably the same.

Step of Forming First Cover Layer and Second Cover Layer

Finally, the first coating agent and second coating agent are dried to evaporate off the adjusting solvent and form the first cover layer and second cover layer. There are no particular restrictions on the drying method, and any drying method can be used. The first coating agent and second coating agent are preferably dried together in a single drying step, but after the first coating agent has been applied, it may be dried before applying the second coating agent.

### EXAMPLES

### Examples 1 to 3

Tablets containing 10 mg of montelukast and placebo tablets were prepared as the tablets of Examples 1 to 3. Each tablet weighed 200 mg and had a rounded shape with a diameter of 8 mm, and the components listed in the “Tablet” column in Table 1 were contained. Each tablet was sprayed with the first coating agent shown in the “First Cover Layer” column in Table 1, and then sprayed with the second coating agent shown in the “Second Cover Layer” column in Table 1. After these two layers were sprayed on, they were dried to produce the laser-printable tablets of Examples 1 to 3. The percentages in parentheses in Table 1 are the weight percent of the component with respect to the total solids content of each cover layer.

TABLE 1

| | | Ingredients | Ex. 1 | | Ex. 2 | | Ex. 3 | |
|---|---|---|---|---|---|---|---|---|
| Tables | | | Placebo Tab. | | Tab. containing | | Tab. containing | |
| | | | with | | Montelukast | | Montelukast | |
| | | | 8 mm-Diameter, | | 10 mg with | | 10 mg with | |
| | | | R-Shape | | 8 mm-Diameter, | | 8 mm-Diameter, | |
| | | | | | R-Shape | | R-Shape | |
| First Cover Layer | Film | Hydroxypropyl- | 3.5 | mg | 3.75 | mg | 3.5 | mg |
| | component | methylcellulose | | | | | | |
| | | Hydroxypropyl | — | | 0.25 | mg | — | |
| | | cellulose | | | | | | |
| | Color | Titanium(IV) oxide | 1.5 mg | | 1.0 mg | | 1.5 mg | |
| | chamge- | | (29.8%) | | (19.9%) | | (29.8%) | |
| | inducing | | | | | | | |
| | oxide | | | | | | | |
| | | Yellow ferric oxide | 0.021 | mg | 0.015 | mg | 0.021 | mg |
| | | Diiron trioxide | 0.0045 | mg | 0.003 | mg | 0.0053 | mg |
| Second Cover Layer | Film | Hydroxypropyl- | 4.9 | mg | 4.65 | mg | 2.45 | mg |
| | component | methylcellulose | | | | | | |

| | | | | |
|---|---|---|---|---|
| Hydroxypropyl | — | 0.25 | mg | — |
| cellulose | | | | |

| | | | | |
|---|---|---|---|---|
| Color | Titanium(IV) oxide | 0.1 mg | 0.1 mg | 0.05 mg |
| chamge- | | (1.999%) | (1.999%) | (1.999%) |
| inducing | | | | |
| oxide | | | | |

| | | | | | | |
|---|---|---|---|---|---|---|
| Yellow ferric oxide | 0.0021 | mg | 0.0015 | mg | 0.0007 | mg |
| Diiron trioxide | 0.00045 | mg | 0.0003 | mg | 0.0002 | mg |

### Comparative Examples 1 to 3

Other than not forming a second cover layer on the laser-printable tablets of Examples 1 to 3 above, the laser-printable tablets of Comparative Examples 1 to 3 were produced in the same manner as in Examples 1 to 3. The numbers in Comparative Examples 1 to 3 correspond to those in Examples 1 to 3.

TABLE 2

| | | Ingredients | Comp. Ex. 1 | | Comp. Ex. 2 | | Comp. Ex. 3 | |
|---|---|---|---|---|---|---|---|---|
| | Tab. | | Placebo Tab. | | Tab. | | Tab. | |
| | | | with | | containing | | containing | |
| | | | 8 mm-Diameter, | | Montelukast | | Montelukast | |
| | | | R-Shape | | 10 mg with | | 10 mg with | |
| | | | | | 8 mm-Diameter, | | 8 mm-Diameter, | |
| | | | | | R-Shape | | R-Shape | |
| First Cover Layer | Film | Hydroxypropyl- | 3.5 | mg | 3.75 | mg | 3.5 | mg |
| | component | methylcellulose | | | | | | |

| | | | | |
|---|---|---|---|---|
| Hydroxypropyl | — | 0.25 | mg | — |
| cellulose | | | | |

| | | | | |
|---|---|---|---|---|
| Color | Titanium(IV) oxide | 1.5 mg | 1.0 mg | 1.5 mg |
| chamge- | | (29.8%) | (19.9%) | (29.8%) |
| inducing | | | | |
| oxide | | | | |

| | | | | | | |
|---|---|---|---|---|---|---|
| Yellow ferric oxide | 0.021 | mg | 0.015 | mg | 0.021 | mg |
| Diiron trioxide | 0.0045 | mg | 0.003 | mg | 0.0053 | mg |

Printability Evaluation

The surface of the laser-printable tablets of Example 1 and Comparative Example 1 were laser printed with a UV laser marking apparatus (model LIS-250D, made by Qualicaps Co., Ltd.) under conditions of PE=53% and 30,000 Hz. FIG. 1a is a photograph of the printing on the laser-printable tablet of Example 1, and FIG. 1b is a photograph of the printing on the laser-printable tablet of Comparative Example 1. It is clear from a comparison of the photographs in FIGS. 1a and 1b that the surface of the laser-printable tablet in Example 1 has been printed more faithfully, whereas it can be seen that the printing is fainter on the surface in Comparative Example 1. This makes it clear that providing a cover layer with a two-layer structure to the surface of the tablet affords better printing on the surface of the tablet, and this is the effect of the present invention.

Evaluation of Whether the Optically Unstable Drug Breaks Down Under UV Laser Irradiation

The surface of the laser-printable tablet of Example 2 was laser printed with a UV laser marking apparatus (model LIS-250D, made by Qualicaps Co., Ltd.) under conditions of PE=53% and 30,000 Hz. The drug contained in the laser-printable tablet before and after printing was analyzed with a high-performance liquid chromatograph (model LC-20A, made by Shimadzu Corp.) to quantify the related substances thereof. The quantification results are shown in Table 3.

TABLE 3

| | Before laser printed | After laser printed |
|---|---|---|
| | with UV laser | with UV laser |
| | (content %) | (content %) |
| Ex. 2 | 0.39 | 0.39 |

As shown in Table 3, in Example 2 there was no change in the related substance content before and after laser printing. This result indicates that providing a cover layer with a two-layer structure to the surface of the tablet suppresses the generation of the related substances by laser irradiation.

Optical Stability Evaluation

The laser-printable tablets obtained in Example 2 and Comparative Example 2 were each put in an optical stability test chamber (model LTB-180C, made by Nagano Science), and were irradiated for one week at a luminance of 2000 lux/hr under a D65 lamp. The drug contained in the laser-printable tablets before and after irradiation was analyzed with a high-performance liquid chromatograph (model LC-20A, made by Shimadzu Corp.) to quantify the related substances thereof. The quantification results are shown in Table 4. The “Before optical irradiation” in Table 4 indicates the content of the related substances before irradiation with light, and the “After optical irradiation” in Table 4 indicates the content of the related substances optical irradiation. The “Amount of increase” indicates the increase in the related substance content resulting from optical irradiation. The lower is the increase in the related substances, the less the optically unstable drug decomposes, that is, the better is the optical stability.

TABLE 4

| | Before optical | After optical | Amount of |
|---|---|---|---|
| | irradiation | irradiation | increase |
| | (content %) | (content %) | (content %) |
| Ex. 2 | 0.39 | 1.29 | 0.90 |
| Comp. Ex. 2 | 0.35 | 1.38 | 1.03 |
| Ex. 3 | 0.18 | 0.50 | 0.32 |
| Comp. Ex. 3 | 0.21 | 1.16 | 0.95 |

As shown in Table 4, the increase in the related substances in Example 2 was less than in Comparative Example 2. Similarly, the increase in the related substances in Example 3 was less than in Comparative Example 3. Also, despite the fact that titanium oxide was contained in the cover layers in a smaller amount in Example 2 than in Comparative Example 3, the increase in the related substances was smaller in Example 2. These results make it clear that providing a cover layer with a two-layer structure to the surface of the tablet suppresses the increase in the related substances upon UV irradiation, and greatly enhances the optical stability of the drug product, and this is the effect of the present invention.

Durability Evaluation

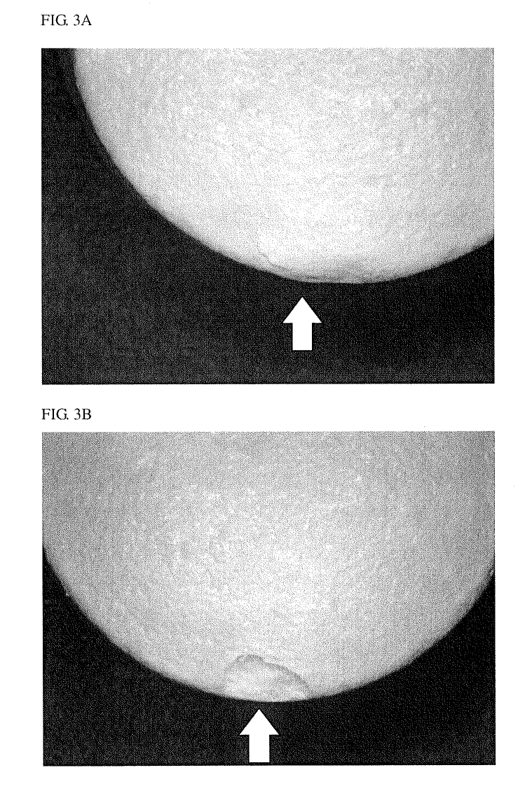

20 of each of the laser-printable tablets of the various working and comparative examples were dropped onto an iron plate from a height of 2 meters, and durability was evaluate from whether or not the tablet surface cracked or chipped. FIG. 3a is a detail photograph of a place where a small crack or chip has formed in the surface of a tablet (light damage), and FIG. 3b is a detail photograph of a place where a more pronounced crack or a chip has formed in the surface of the tablet (heavy damage). As a result of evaluating the durability of the above-mentioned 20 tablets, the number of tablets in which no damage occurred, the number in which cracking occurred to a similar degree as the light damage shown in FIG. 3a, and the number that in which cracking occurred to a similar degree as the heavy damage shown in FIG. 3b are given in Table 5 below. The greater is the number of instances of light and heavy damage, the lower is the durability.

TABLE 5

| | No Damage | Light Damage | Heavy Damage |
|---|---|---|---|
| Ex. 1 | 20 | 0 | 0 |
| Ex. 2 | 20 | 0 | 0 |
| Ex. 3 | 20 | 0 | 0 |
| Comp. Ex. 1 | 11 | 7 | 2 |
| Comp. Ex. 2 | 16 | 4 | 0 |
| Comp. Ex. 3 | 10 | 9 | 1 |

As shown in Table 5, in Examples 1 to 3 no cracking or chipping occurred on the tablet surface, whereas in Comparative Examples 1 to 3, cracking and chipping did occur in the tablet surface. These results show that providing a two-layer coating, namely, the first cover layer and the second cover layer, makes it less likely that cracking and chipping will occur on the tablet surface, and this is the effect of the present invention.

### REFERENCE SIGNS LIST

1 tablet
2 first cover layer
3 second cover layer

1. A laser-printable tablet comprising:
a tablet containing an optically unstable drug,
a first cover layer that covers the tablet and contains a first color change-inducing oxide, and
a second cover layer that covers the first cover layer and contains a second color change-inducing oxide,
the concentration in which the second color change-inducing oxide is contained in the second cover layer being different from the concentration in which the first color change-inducing oxide is contained in the first cover layer.

2. The laser-printable tablet according to claim 1, wherein
the first cover layer contains the first color change-inducing oxide in an amount of at least 10 wt % and no more than 50 wt %, and
the concentration in which the second color change-inducing oxide is contained in the second cover layer is lower than the concentration in which the first color change-inducing oxide is contained in the first cover layer.

3. The laser-printable tablet according to claim 1, wherein
the second cover layer contains the second color change-inducing oxide in an amount of no more than 15 wt %.

4. The laser-printable tablet according to claim 1, wherein
the first cover layer contains the first color change-inducing oxide in an amount of at least 15 wt % and no more than 30 wt %.

5. The laser-printable tablet according to claim 1, wherein
the optically unstable drug is one or more types selected from the group including of olanzapine, montelukast sodium and rosuvastatin.

6. A method for manufacturing a laser-printable tablet comprising steps of:
covering the surface of a tablet containing an optically unstable drug with a first coating agent containing a first color change-inducing oxide,
covering the surface of the first coating agent with a second coating agent containing a second color change-inducing oxide whose concentration is different from that of the first color change-inducing oxide, and
drying the first coating agent and the second coating agent, thereby forming a first cover layer and a second cover layer in that order on the surface of the tablet.

7. The method for manufacturing a laser-printable tablet according to claim 6, wherein
the first coating agent contains a color change-inducing oxide in an amount of at least 10 wt % and no more than 50 wt % with respect to the total solids content, and
the concentration of the second color change-inducing oxide contained in the second coating agent is lower than the concentration of the first color change-inducing oxide contained in the first coating agent.
